(19) United States
(12) Patent Application Publication
(10) Pub. No.: US 2003/0204150 A1
(43) Pub. Date: Oct. 30, 2003

(54) Method and apparatus for the 3-dimensional analysis of movement of the tooth surfaces of the maxilla in relation to the mandible

(76) Inventor: Wolfgang Brunner, Maierhofen (DE)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/423,424

(22) Filed: Apr. 25, 2003

(30) Foreign Application Priority Data
Apr. 25, 2002 (DE) 102 18 435.6

(51) Int. Cl. A61B 5/103; A61B 5/117

(52) U.S. Cl. 600/590

(57) ABSTRACT

A method and apparatus are disclosed for the 3-dimensional analysis of movement of tooth surfaces of the maxilla (MX) in relation to the mandible (MN), comprising a measurement system for determining the movement of two solid bodies in all degrees of freedom by means of electronic measurement sensors. A measurement MX-sensor is mounted with respect to the maxilla and a measurement MN-sensor is mounted with respect to the mandible, and the positions of the measurement sensors are recorded continuously during the performance of mandible movements.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention relates to a method and apparatus for the 3-dimensional analysis of the movement of the tooth surfaces of the maxilla (MX) in relation to the mandible (MN).

### BACKGROUND OF THE INVENTION

Digitization of the surfaces of tooth areas is a known procedure. It is a known principle that the movement of these tooth surfaces with respect to one another can be represented. In the patent U.S. Pat No. 6,152,731 a method is described with which the surfaces of the maxillary and the mandibular teeth can be digitized and placed in relation to one another by means of joint axes. It is likewise known from the patent DE 35 74 340 that mandibular movements can be measured in all degrees of freedom and the positional data recorded by means of a computer unit. Furthermore, DE 35 74 340 discloses a way to specify measurement points in the tooth-surface region three-dimensionally by means of a probe tip.

However, in order to reconstruct the movement of tooth surfaces associated with the maxilla with respect to those of the mandible it is necessary to establish the relation of the sensors in the system for measuring the movement, i.e. the MX-sensors relative to the maxilla and the MN-sensors to the mandible, a process which either is not described or is very laborious to implement.

### BRIEF SUMMARY OF THE INVENTION

The object of the invention is to enable an electronic movement-measuring system, employing electronic sensors that can determine the relative positions of at least two solid bodies in all degrees of freedom, to be used to represent the tooth surfaces of the maxilla with respect to the mandible in such a way that the relation between the sensors at the maxilla and the tooth surface on the mandible and the sensory system associated with the mandible and the tooth surface of the mandible and the relation of maxilla to mandible can be determined, and hence to enable the movement of the tooth surfaces of the maxilla with respect to tooth surfaces of the mandible to be represented and analyzed by a simple, easily implemented procedure.

According to a first aspect of the present invention there is provided a method for the 3-dimensional analysis of movement of tooth surfaces of the maxilla (MX) in relation to the mandible (MN), comprising a measurement system for determining the movement of two solid bodies in all degrees of freedom by means of electronic measurement sensors, wherein a measurement MX-sensor is mounted with respect to the maxilla and a measurement MN-sensor is mounted with respect to the mandible, and the positions of the measurement sensors are recorded continuously during the performance of mandible movements, wherein the improvement comprises:

in relation to the MX-sensor, at least three maxilla-referential points are measured, which are at one of the following three locations:

i. directly apposed to the surfaces of the maxilla teeth,

ii. situated on or at an accessory that is connected to the maxilla teeth,

iii. distinguished by having a fixed geometrical relation to the MX-sensor and the maxilla teeth;

and in relation to the MN-sensor, at least three mandible-referential points are measured, which are at one of the following three locations:

i. directly apposed to the surfaces of the mandible teeth,

ii. situated on or at an accessory that is connected to the mandible teeth,

iii distinguished by having a fixed geometrical relation to the MN-sensor and the mandible teeth;

the maxilla- and mandible-referential points having a predetermined spatial relationship to one another at at least one point in time during the measurement;

by means of at least one electronic surface-digitization unit at least partial areas of at least one tooth in the maxilla and one tooth in the mandible are measured by one of intraoral inspection and consideration of previously constructed tooth models;

and by means of a computer unit and display unit the digitized tooth surfaces are brought into relation with the positions of the maxilla- and mandible-referential points and from the recorded tooth movements are derived digitized representations of the tooth areas in their movement with respect to the MX-sensor and the MN-sensor.

According to a second aspect of the present invention there is provided an apparatus for the 3-dimensional analysis of movement of tooth surfaces of the maxilla (MX) in relation to the mandible (MN) using the method according to the first aspect of the present invention and comprising a measurement MX-sensor adapted for mounting with respect to a maxilla, a measurement MN-sensor adapted for mounting with respect to a mandible, an electronic surface-digitization unit by means of which at least partial areas of at least one tooth in the maxilla and one tooth in the mandible can be measured by one of intraoral inspection and consideration of previously constructed tooth models, and a computer unit and display unit whereby the digitized tooth surfaces are brought into relation and digitized representations of the tooth areas in their movement with respect to the MX-sensor and the MN-sensor can be obtained.

A position indicator sensor in the form of a stylus can be used to mark points that are anatomically significant with respect to the MX- and MN-sensors, or points having a fixed, geometrical relation to anatomically significant structures, which can serve as reference points in the digitized representation of tooth surfaces, or have a specific relation thereto, and can be brought into register therewith. Instead of the tooth surfaces, any other solid or fixed structures in the jaws can be used, such as the fixed gum tissue (gingiva), sections of the palate or toothless jaw sections when no stably positioned teeth are present.

In this process it will sometimes be appropriate to calculate the position of the scanning stylus from the positions of the measurement sensor apparatus determined by the movement-measuring system.

In principle, the movement-measuring system can be variously designed. For instance, the position of the scanning stylus in relation to the maxilla can be measured directly with reference to the MX-sensor. This possibility can be employed when the determination of position is being implemented, for example, by measuring the transit time of ultrasound pulses. However, it is also possible to detect position by means of external measurement recorders; optical recorders such as a camera can be used for this purpose.

The selected points on the tooth surfaces should be anatomically significant or have a specific geometrical relation to anatomically significant structures, and can be visualized together with the digitized tooth surfaces on a computer's display unit, where the two representations can be brought into optimal register with one another. By using redundant points, where appropriate the accuracy can be increased. The digitized tooth surfaces can be generated indirectly, i.e., not from the actual dentition but from previously constructed tooth models. For this purpose plaster impressions are made, from which positive casts can be produced to simplify the digitization. Digitization of these positive casts can be done, for example, with laser or strip-light scanners.

Instead of the scanning stylus, an intraoral digitization unit can be employed in some embodiments of the invention. To this unit, as an analog to the stylus tip, there is connected in a specified manner a measurement sensor apparatus for the 3D determination of position. With this arrangement one or more partial tooth areas can be scanned, which for instance can be brought into register with a digitized representation of the whole tooth area.

In one embodiment of the invention a relationship between the tooth impressions of the maxilla and the maxillary sensor can be established. Here, the maxillary sensor can be attached directly, by means of an accessory, to the teeth of the maxilla. In a particularly advantageous embodiment the MX-sensor is attached to a facebow situated in a specified, fixed relation to the maxilla. The MX or MN sensor can be placed extraoral in this way. It is also possible to place the sensors intraoral to the teeth of the maxilla (MX) and to the teeth of the mandible (MN). For that, miniaturized sensors have to be used.

Depending on which movement-measuring system is used, the maxillary sensor can comprise active measurement sensors, which can be constructed for instance as an optical 3D-display unit with several electronic cameras; in this case, the resulting data can be sent to a computer unit for calculation of the three-dimensional positions of the mandibular sensors.

The MN-sensors in turn may comprise either passive reflectors (markers) or active LED markers.

In another embodiment the MX- and MN-sensors may comprise passive reflectors or active LED markers, with the corresponding recording unit in the form of electronic cameras mounted on spatially fixed holders.

In other embodiments the MX- and MN-sensors operate according to the principle of measuring the transit times of ultrasound pulses, by means of magnetic fields or by means of laser technology.

A bite plate, that may be in the form of a bite fork, can be provided and covered on one side with a deformable recording substance, e.g. in the form of wax, ZnO paste, Kerr stick or the like. At the same time the bite plate comprises markings, the 3D positions of which with respect to the MX-sensor can be input by means of a 3D scanning stylus.

The bite plate is anchored to the upper jaw by way of the recording substance, and at least three marker points are input by means of the scanning stylus.

In a particular embodiment, the bite plate is provided with a holder in which can be attached a sensor for the detection of all positional degrees of freedom. In this case it is especially advantageous to use the MN-sensor, which would be needed in any case. The holder must be so constructed that the 3D-position sensor can be attached as specified and cannot be displaced. It is particularly advantageous here to use magnetic or mechanical fasteners, or snap fasteners, that are simple and can be rapidly closed and opened.

After the bite plate has been anchored to the maxilla by way of the recording substance, the position of the 3D-position sensor with respect to the MX-sensor is determined.

The impressions made in the recording substance can be detected by means of a 3D-digitization unit. These data can be collected directly or indirectly: indirectly, when a model is made by pouring a casting material into the impressions, and then that model is scanned in.

These digitized data can be used directly to represent the tooth areas in movement. A potential disadvantage of this approach, however, is that the tooth surfaces are not sufficiently complete. Hence it is possible, in a separate process, to make tooth impressions, e.g. in a mass of plaster. The surface of these plaster impressions, or of a positive cast made from them, can then be digitized and brought into the best possible register with the surface of the recording substance on the bite plate, by means of the computer unit.

In order to establish a relation between MX-sensor and maxilla, the marker points must also be visible in the digitized surface, or must be situated in a specified, reproducible geometrical relation to anatomical structures. As a result, these can be brought into register with the measured points in the computer, so that the relation can be established.

Similarly, the holder must bear features that enable the establishment of such a relation.

In another embodiment, extra marker points can be provided, which can be unambiguously identified after digitization and which have a fixed relation to the points as aforesaid or the holder or MX-sensor. These points may comprise, for example, notches or depressions on the bite plate or other kinds of markings.

In accordance with the invention, at at least one point in time the relation of the maxilla to the mandible must be determined.

In the method, a recording medium, e.g. wax, is applied to the upper and lower surfaces of the bite plate. Now the teeth of both the maxilla and the mandible are anchored to the bite plate by pressing them into the recording medium. To the mandible, preferably to the mandibular teeth, the MN-sensor is now attached by means of a holder. In this position at least one measurement of the position of the mandible is carried out. To enable unhindered movement of the teeth, the attachment to the mandible should be para-occlusal.

The position of the maxilla with respect to the MX-sensor can be established in the same way.

If a recording substance is produced for making a conventional impression to show the position of the maxilla with respect to the mandible, this substance can also be fixed to the bite plate intraorally or combined with another layer (e.g., plaster) such that only the mandible tooth rows become detached from the recording substance, which remains stuck to the maxilla teeth or is repositioned thereon. The MN-sensor, attached by way of the bite plate establishes the relation of the recording substance, with its maxilla and mandible teeth impressions, to the upper sensor. From the position of the recording substance (which as a rule is of the intercuspidation type) the mandible movements can be determined.

In an especially advantageous embodiment, the device for attaching the MN-sensor to the mandible comprises the same holder as does the bite plate. As described above, this can be a magnetic or mechanical fastener. The advantage is that the same sensor can be used on the bite plate and at the mandible, and rapid exchange can be accomplished in a single operation.

Thus, for example, the sensor is first fixed to the bite plate. The teeth of the maxilla and the mandible are seated in the impressions. Now a position measurement is carried out. The teeth remain in the impressions. Thereafter, the same sensor can be attached to the device on the mandible and a new position measurement can be carried out. The advantage resides in the rapidity with which the position of the sensor can be changed, and in the simplicity of construction.

After these preparations have been completed, the actual movement measurement can be begun. For this purpose the bite plate is removed.

The movements must now be executed with the MN-sensor in the same relation to the mandible as it was for the position measurement using the bite plate.

On both sides of the bite plate there must be marker points that allow a relationship between the maxilla and the mandible surfaces to be established after digitization. The marker points must be identifiable after digitization and can, for example, take the form of bores in or elevations on the bite plate.

As described above for the tooth surfaces of the maxilla, in accordance with the invention it is possible first to produce dentition impressions and where appropriate positive models thereof, which after surface-digitization by means of a computer unit are brought into the best possible register with the digitized surfaces of the impressions made in the recording substance on the bite plate. However, this requires extra effort.

Preferably, the layers of recording substance on the bite plate should be so thick that the side surfaces of the teeth also make an impression. In some cases the recording substance on both sides of the bite plate can be shaped, for example into a trough. In some cases an aperture must be present in the mandibular trough, to leave room for the holder by which the MN-sensor is attached.

In a further modification, it is possible to use intraoral digitizing units instead of making plaster impressions. These can operate, for example, on the basis of laser scanning or by strip-light projection.

The various aspects of the present invention will now be described by way of example with reference to the accompanying drawings.

### BRIEF DESCRIPTION OF THE SEVERAL VIEWS OF THE DRAWINGS

FIG. 1 is a flow diagram of a preferred form of measurement procedure;

FIG. 2 is a flow diagram of the steps in an associated evaluation procedure;

FIG. 3a shows the arrangement of an MX-sensor and bite plate;

FIG. 3b shows the arrangement of an MX-sensor and an MN-sensor;

FIG. 3c shows the arrangement of a position-sensing apparatus during the measurement procedure;

FIG. 4 is a perspective view of an exemplary embodiment of a position sensor;

FIG. 5a is a plan view of an exemplary embodiment of a bite plate with markings;

FIG. 5b is a side view of an exemplary embodiment of a bite plate with markings;

FIG. 6 is a perspective view of a preferred embodiment of bite plate.

### DETAILED DESCRIPTION OF THE INVENTION

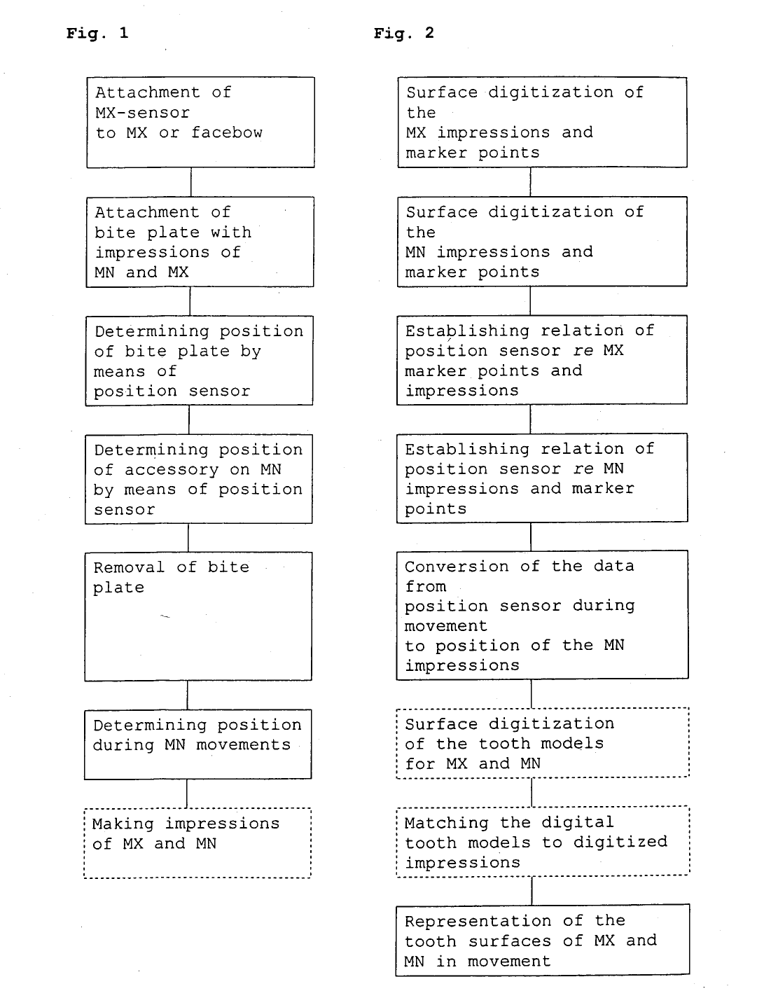

In FIG. 1 a preferred and especially simple form of measurement procedure is set out. Here a bite plate with recording substance on both sides should be used. The production of supplementary impressions, for example from plaster, is optional.

FIG. 2 shows the steps needed for evaluation.

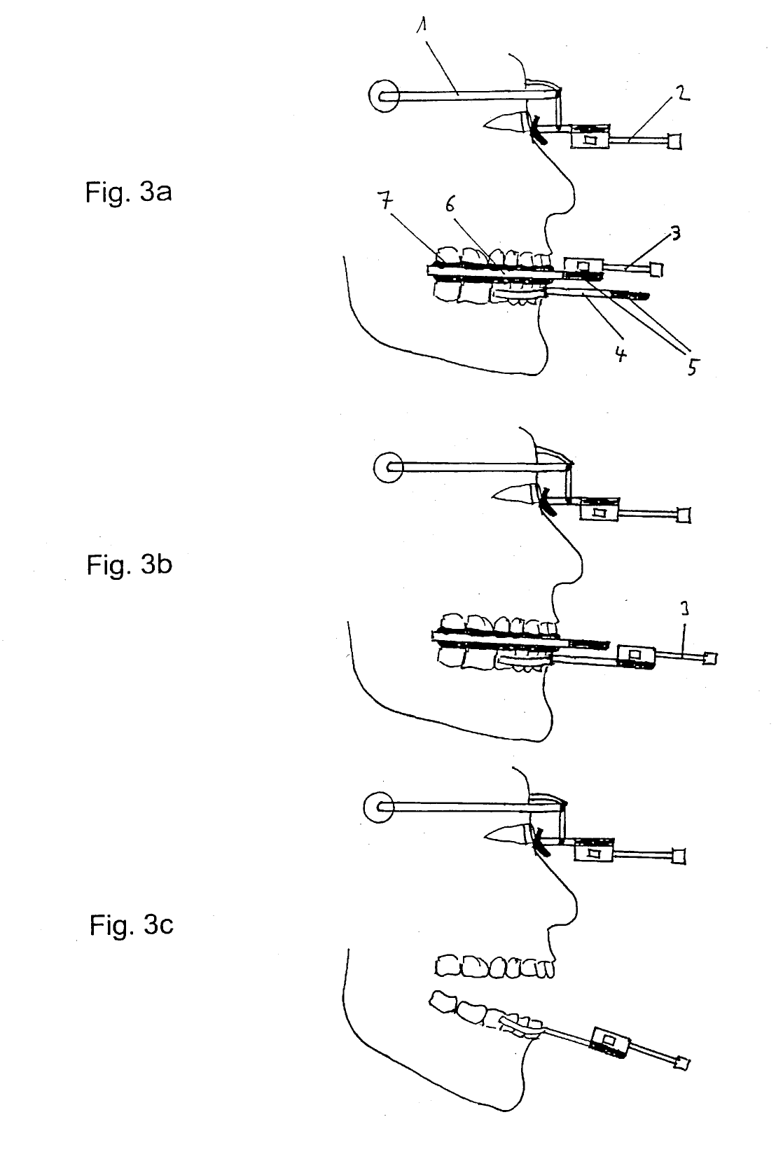

In FIG. 3a is shown a measurement arrangement that comprises a facebow 1, an MX-sensor 2, a position sensor 3, a sensor holder for MN 4 with holding device 5, a bite plate 6, which may take the form of a bite fork, and deformable recording substance. When the measurement sensors are arranged as shown here, the position of the bite plate is determined.

In FIG. 3b the position sensor 3 is used as a mandibular sensor. With this arrangement at least one positional measurement is carried out.

FIG. 3c shows the arrangement for carrying out the measurement. When position sensors are used that employ the principle of measuring the transit times of ultrasound pulses, the position sensor 2 can consist of three measurement microphones and the position sensor 3 of three ultrasound transmitters.

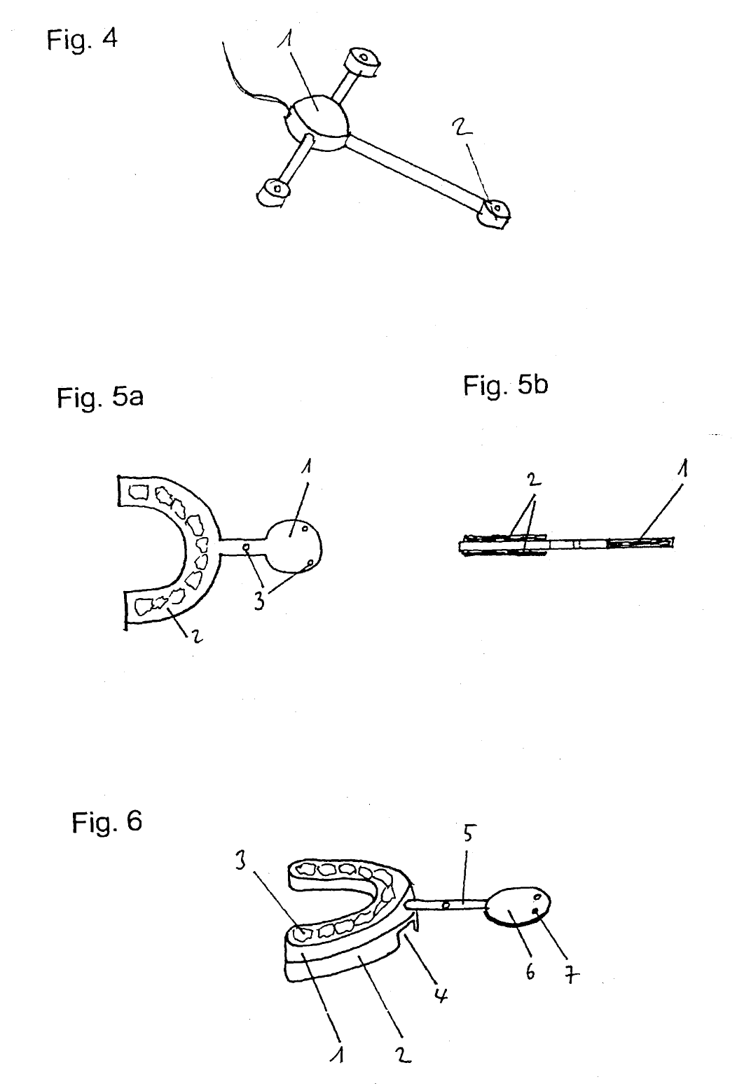

FIG. 4 shows an exemplary embodiment of a position sensor with a device for attachment to a measurement holder 1 and with the measurement sensors 2.

FIG. 5a shows a bite plate in plan view, comprising a base plate with recording substance 2 on both sides, a holding device for the position sensor 1 and the digitizable position points 3 on both sides. FIG. 5b shows the bite plate in side view.

In FIG. 6 is shown a preferred embodiment of a bite plate. This consists of an upper and lower trough 1 and 2 filled with recording substance 3, and allows impressions to be made that show the structure of the sides of the teeth. An aperture 4 is provided, at which the holding device for the MN-sensor can be disposed. The points 5 and 6 show the device to which a position sensor can be attached. Point 7 represents one of the marker points, which have a fixed spatial relation to the tooth impressions and also enable arrangement of the upper side with respect to the lower side of the bite plate.

1. Method for the 3-dimensional analysis of movement of tooth surfaces of the maxilla (MX) in relation to the mandible (MN), comprising a measurement system for determining the movement of two solid bodies in all degrees of freedom by means of electronic measurement sensors, wherein a measurement MX-sensor is mounted with respect to the maxilla and a measurement MN-sensor is mounted with respect to the mandible, and the positions of the measurement sensors are recorded continuously during the performance of mandible movements, wherein the improvement comprises:
in relation to the MX-sensor, at least three maxilla-referential points are measured, which are at one of the following three locations:
i. directly apposed to the surfaces of the maxilla teeth,
ii. situated on or at an accessory that is connected to the maxilla teeth,
iii. distinguished by having a fixed geometrical relation to the MX-sensor and the maxilla teeth;
and in relation to the MN-sensor, at least three mandible-referential points are measured, which are at one of the following three locations:
i. directly apposed to the surfaces of the mandible teeth,
ii. situated on or at an accessory that is connected to the mandible teeth,
iii distinguished by having a fixed geometrical relation to the MN-sensor and the mandible teeth;
the maxilla- and mandible-referential points having a predetermined spatial relationship to one another at at least one point in time during the measurement;
by means of at least one electronic surface-digitization unit at least partial areas of at least one tooth in the maxilla and one tooth in the mandible are measured by one of intraoral inspection and consideration of previously constructed tooth models;
and by means of a computer unit and display unit the digitized tooth surfaces are brought into relation with the positions of the maxilla- and mandible-referential points and from the recorded tooth movements are derived digitized representations of the tooth areas in their movement with respect to the MX-sensor and the MN-sensor.

2. Method as claimed in claim 1, wherein at least three maxilla-referential points are determined in relation to the MX-sensor by using a position-indicator sensor to sample at least three marker points located substantially at one of the tooth surfaces of the maxilla and at points in a specified or specifiable geometry, and are measured three-dimensionally with respect to the MX-sensor, and that these points are selected such that they are identified in the electronically digitized surfaces and can be brought into register.

3. Method as claimed in claim 1, wherein at least three mandible-referential points in relation to the MN-sensor and to the MX-sensor are determined by keeping the MN-sensor in a fixed relationship to the mandible and measuring its position with respect to the maxilla continuously, while simultaneously using a position-indicator sensor to sample at least three marker points located substantially at the tooth surfaces of the mandible, which are measured three-dimensionally with respect to the MN-sensor.

4. Method as claimed in claim 1, wherein a position-indicator sensor comprising at least one of mechanical sensing apparatus and optical sensing apparatus for surface measurement is provided, with which at least one tooth surface of the maxilla and the mandible can be detected that, by means of the computer unit, can either be represented while in movement or brought into register with a larger digitized tooth area.

5. Method as claimed in claim 1, wherein the relationship of the MX-sensor to at least three maxilla-referential points is determined by the following steps:
keeping an accessory in the form of a bite plate in fixed relation to the teeth of the maxilla by way of dental impressions in a deformable layer borne thereon, there being at least three marker points on the accessory that are distinguished by having a fixed geometrical relationship to the maxilla teeth and to the MX-sensor, and
displaying the marker points by means of a position-indicator sensor and hence determining the position of the marker points with respect to the MX-sensor.

6. Method as claimed in claim 5, wherein on the bite plate, instead of the marker points there is an apparatus to which a position sensor of the type of the MN-sensor can be connected in a fixed geometry and can be used to determine the position of the accessory with respect to the MX-sensor.

7. Method as claimed in claim 5, wherein on the bite plate at least three marker points are disposed that are located in a fixed relation to all other markings and to the dental impressions, and which can be detected by an electronic digitization unit in order to determine the relationship between tooth areas and the MX-sensor.

8. Method as claimed in claim 1, wherein the maxilla- and mandible-referential points are determined at at least one point in time, by employing a deformable recording layer on the upper and lower sides of an accessory in the form of a bite plate to obtain simultaneous impressions of tooth areas of the maxilla and the mandible, and in addition the position of the MN-sensor with respect to the MX-sensor is recorded.

9. Method as claimed in claim 1, wherein an accessory in the form of a bite plate is used to obtain simultaneous impressions of tooth areas of the maxilla and the mandible, which bite plate is fixedly connected to a conventional impression-recording substance and the position of the impression-recording substance above the MN-sensor with respect to the MX-sensor is recorded.

10. Method as claimed in claim 8, wherein the bite plate is used to determine the relationship of the MX-sensor to tooth surfaces of the maxilla and the relationship of tooth surfaces of the maxilla to tooth surfaces of the mandible by keeping the accessory in fixed relation to the teeth of the maxilla by way of dental impressions in the deformable layer borne thereon, while the MN-sensor is mounted in fixed relation to the mandible, and at least one measurement is made by the MN-sensor in the position in which the teeth of the maxilla and mandible are seated in the impressions in the bite plate.

11. Method as claimed in claim 8, wherein the MN-sensor is attached to the mandible and on the basis of the relationship between the maxilla and the mandible established by the bite plate recording substance, the mandible movements are recorded.

12. Method as claimed in claim 8, wherein both at the bite plate and also at the attachment by means of which the MN-sensor is mounted, a means is provided that allows the MN-sensor to be moved from the bite plate to an MN-sensor holder, so that one and the same sensor can be used.

13. Method as claimed in claim 10, wherein the recording of the mandible movement is carried out after removal of the bite plate, while the MN-sensor remains in the same position.

14. Method as claimed in claim 8, wherein the bite plate comprises markings on both sides that enable the impressions of the maxilla and the mandible to be brought into an unambiguous relation to one another by means of an electronic surface-digitizing unit.

15. Method as claimed in claim 8, wherein the deformable recording layer in which the dental impressions are made is so thick that the areas on the sides of the teeth are represented, so that substantially complete tooth surfaces can be observed in movement.

16. Method as claimed in claim 5, wherein the at least partial areas of at least one tooth in the maxilla and one tooth in the mandible measured by one electronic surface-digitization unit are put in relation to one another by means of the computer unit with reference to three digitized markers that are related in a specified manner to the maxilla and the mandible, and that these are put in relation to the MX-sensor with reference to the identical or additional digitized points, and that these are put in relation to the MN-sensor for the point in time at which the maxilla and the mandible are seated in the deformable layer, and that during the movement of the mandible these are represented by means of the display unit in individual movement phases, for the same relation of the MN-sensor to the mandible.

17. Method as claimed in claim 1, wherein additional models of teeth of the maxilla and the mandible are made and electronically digitized and, by means of the computer unit, at least partial areas of the additional tooth surfaces are fixed in position by putting them in the best possible register with the digitized tooth areas already obtained so that they can be completed and extended.

18. Apparatus for the 3-dimensional analysis of movement of tooth surfaces of the maxilla (MX) in relation to the mandible (MN) comprising a measurement MX-sensor adapted for mounting with respect to a maxilla, a measurement MN-sensor adapted for mounting with respect to a mandible, an electronic surface-digitization unit by means of which at least partial areas of at least one tooth in the maxilla and one tooth in the mandible can be measured by one of intraoral inspection and consideration of previously constructed tooth models, and a computer unit and display unit whereby the digitized tooth surfaces are brought into relation and digitized representations of the tooth areas in their movement with respect to the MX-sensor and the MN-sensor can be obtained.

19. Apparatus as claimed in claim 18, wherein a position-indicator sensor is provided to sample at least three marker points located substantially at one of the tooth surfaces of the maxilla and at points in a specified or specifiable geometry.

20. Apparatus as claimed in claim 18, wherein a position-indicator sensor is provided that comprises one of mechanical sensing apparatus and optical sensing apparatus for surface measurement with which at least one tooth surface at the maxilla and the mandible can be detected and which by means of the computer unit is represented while in movement, or which is brought into register with the digitized tooth area.

21. Apparatus as claimed in claim 18, wherein a position-indicator sensor and an accessory in the form of a bite plate are provided in order that the relationship of the MX-sensor to at least three maxilla-referential points can be determined by the following steps:
keeping the bite plate in fixed relation to the teeth of the maxilla by way of dental impressions in a deformable layer borne thereon, there being at least three marker points on the accessory that are distinguished by having a fixed geometrical relationship to the maxilla teeth and to the MX-sensor, and
displaying the marker points by means of the position-indicator sensor and hence determining the position of the marker points with respect to the MX-sensor.
